(19) United States
(12) Patent Application Publication
(10) Pub. No.: US 2021/0121161 A1
(43) Pub. Date: Apr. 29, 2021

(54) SALIVA COLLECTION AND TESTING SYSTEM FOR PREDICTIVE MEDICINE

(71) Applicant: AICUBE S.R.L., Trezzano sul Naviglio (Ml) (IT)

(72) Inventor: Stefano Russano, Trezzano sul Naviglio (MI) (IT)

(21) Appl. No.: 17/257,067

(22) PCT Filed: Jul. 1, 2019
(86) PCT No.: PCT/IB2019/055598

§ 371 Date: Dec. 30, 2020

(30) Foreign Application Priority Data
Jul. 4, 2018 (IT) 102018000006925

(51) Int. Cl. A61B 10/00; A61C 17/22; A61C 17/34; A61C 17/06; A61C 17/26; A61B 5/1172; A46B 5/00; A46B 9/04; A46B 13/02; A61B 5/021; A61B 5/026; A46B 15/00; A61B 5/00

(52) U.S. Cl. CPC A61B 10/0051; A61C 17/221; A61C 17/3436; A61C 17/065; A61C 17/26; A61B 5/1172; A61B 2560/0456; A46B 9/04; A46B 13/02; A61B 5/02141; A61B 5/026; A46B 15/0053; A61B 5/0022; A46B 5/0095

(57) ABSTRACT

A saliva collection and testing system comprising an electric toothbrush (1) suitable for taking a user's saliva by way of taking means (15), and a test unit (21) for testing the taken saliva, said test unit (21) including a seat (22) for the electric toothbrush (1) comprising means (25, 33) for taking the saliva collected by the toothbrush, said test unit (21) cleaning-up the saliva taking means (15) of the electric toothbrush (1) after saliva is taken so as to prepare said electric toothbrush (1) to a subsequent use. A digital tooth fingerprint sensor is present on the toothbrush and allows to recognize the user. Advantageously the test results are sent to a remote unit via a wireless system and in an encrypted mode for their subsequent processing by one or several Machine Learning algorithms which exploit an Artificial Intelligence.

The scope of the present invention is a saliva collection and testing system for predictive medicine, according to the preamble of the main claim.

The investments in the health system reached unsustainable levels for most developed countries and continually grow exponentially. However, not always the value of the investments as referred to the GDP of the States corresponds to a better offer of health services to citizens. All governments in the world are thus looking for new forms of investment that introduce innovations in the public health service field and lead to reduced public costs. Many analysts of the public health service sector indicate that the use of artificial intelligence might be one of the most promising solutions to go in this direction.

Also, the need for reducing cares moves jointly with the possibility of making an early diagnosis of diseases through prevention and predictive medicine. However, this requires a constant collection of homogeneous data on distinctive parameters that might provide every specific user with guidelines on lifestyle or on cares, earlier than with the present methodologies.

Devices are known suitable for assessing the physical-health conditions of users, such as, for instance, sensors capable of assessing and measuring the heartbeat and the blood flow while an aggregated data collection device is being used.

Devices are also known suitable for taking a measurement of the cortisol level in a user's saliva. For instance, U.S. Pat. No. 6,833,274 discloses how such device overcomes the drawbacks of other systems by providing sensors that are capable of measuring the cortisol levels in a sample in real time, while also offering high selectivity, high sensitivity, ease of use, low cost, and portability advantages. The terms “real time measurement of cortisol levels” or “real time” relate to a relatively quick assessment of the cortisol level in a sample of a fluid just produced by a body so as to indicate present cortisol level data.

Note that cortisol is a steroid hormone produced by adrenal glands: a cortisol level exceeding the normal one might indicate the presence of the severe diseases such as, for example, a Cushing syndrome or a tumor of adrenal glands. An excessive cortisol level is also associated with stress situations, severe hepatic diseases or depression.

Devices are also known suitable for taking a measurement of the α-amylase level in saliva. As a matter of fact, it is known that production of α-amylase in salivary glands increases as a response to a psychological and physical stress through interactions with the autonomic nervous system. Controlling such enzyme has been proved to be useful as an activity indicator in the autonomic nervous system. For example, JP2002168860A discloses a methodology capable of measuring variations of α-amylase in saliva, whereas document US2011/0040164A1 discloses a technology for producing a sensor that can be used in a testing system.

Other known solutions that specify the use of a toothbrush for collecting a user's saliva so as to submit such sample to a testing and to assess any user's diseases are known from US2002/127143, US2017/135464, WO2015/073062, and US2015/088538.

An object of the present invention is to offer a system that enables a precisely identified user to submit his/her own saliva to medical tests after every use of an electric toothbrush in a simple and substantially automatic manner.

Specifically, an object of the present invention is to offer a system of the mentioned type that is simple to use by a user and allows to take saliva for submitting it to a test without any need for such user to actively operate onto such sample.

Another object is to offer a system of the mentioned type that is capable of autonomically and automatically recognizing the user who performs the taking of saliva and is able to associate the automatically performed test with such user even when the toothbrush is used by several people (provided the part in contact with the mouth is replaced, indeed).

A further object is to offer a system that can operate a Machine Learning Analysis of the medical data extracted from a user's saliva, by way of a comparison even with information continually updated via the Internet.

Another object is to offer a system of the mentioned type that features reduced dimensions.

These objects and others which will be apparent to those skilled in the art are achieved by a saliva collection and testing system for predictive medicine according to the main claim.

The following drawings are attached for explanatory, non-limitative purposes for a better understanding of the present invention, wherein:

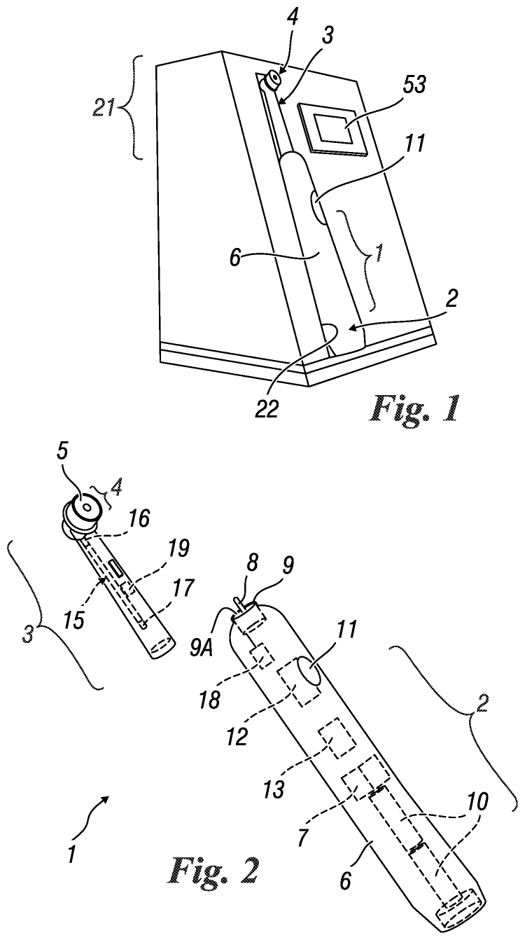

FIG. 1 shows a perspective view of a system according to the invention;

FIG. 2 shows an exploded view of a portion of the system of FIG. 1;

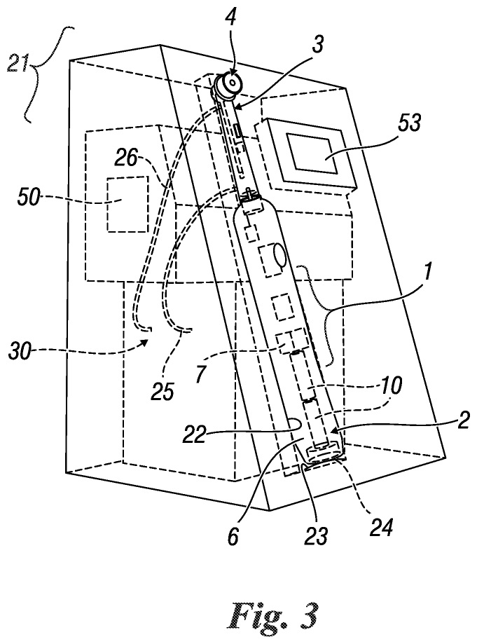

FIG. 3 shows a transparent perspective view of the system of FIG. 1;

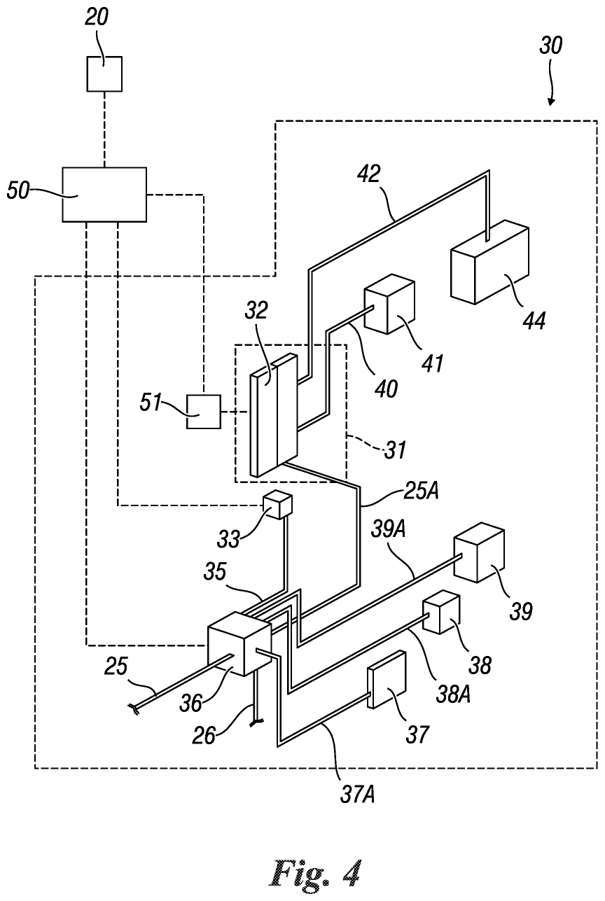

FIG. 4 shows a schematic perspective view of a portion of the system of FIG. 1; and

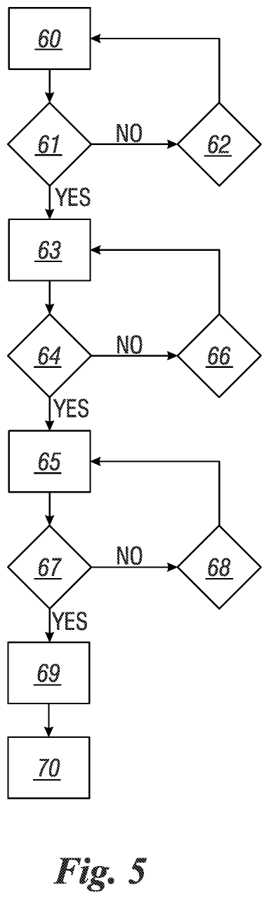

FIGS. 5 thru 9 show flow diagrams representing different operating steps of the system of FIG. 1.

With reference to the mentioned figures, a system according to the invention comprises an electric toothbrush 1 including a base portion 2 and a preferable separatable accessory 3 including a usual rotary body 4 with bristles 5 suitable for allowing to brush the user's teeth. The base portion 2 comprises a usual body 6 containing a motor 7 for driving the rotary body 4 into rotation (in a known manner) whenever the separatable accessory 3 is associates with a usual rotary shaft 8 projecting from a seat 9A provided at an end 9 of the base portion 2 where the accessory 3 is inserted.

The latter also comprises (rechargeable or non-rechargeable) internal batteries 10 and, according to the invention, a fingerprint sensor 11 connected to a control and communication device 12, of a wireless or Bluetooth type, complete with a receiver 20 (FIG. 4) associated with a test or base unit 21 which will be described below. The sensor 11 (by way of its own electronics or via devices 12 and/or 20) allows to associate the reading of a digital fingerprint with a specific user (user ID) previously stored in the base unit 21 in order to authorize a subsequent taking of a saliva sample and to get a unique and automatic sample/user association.

If a user is not recognized, then the use of the system according to the invention is prevented.

The fingerprint can also be read under non-optimum conditions, in the presence of liquids (water, soap, etc.), creams, or other substances. In addition, advantageously the sensor 11 is of a type capable of also measuring a user's heartbeat (and consequently also his/her blood pressure and flow).

In addition, the base portion 2 of the electric toothbrush also includes a movement sensor 13, of a known type whatsoever, suitable for assessing the movement (acceleration and position in the space) of the electric toothbrush 1 with respect to the base unit 21 (which accommodates it for recharging purposes, as better described below). Preferably, such sensor 13 is also connected to the above-mentioned device 12. Finally, the base portion 2 also contains a micropump 18 monitored and controlled by the device 12 and connected to the seat 9A for the accessory 3 in an inherently known manner.

The separatable accessory 3 is provided with a collection chamber 15 for collecting a user's saliva sample: such collection chamber is defined, for example, by a channel realized along such accessory 3. The collection chamber or channel 15 is provided with two preferably extremal “push-up” valves (16 and 17), for instance made from polydimethylxyloxane (PDMS), which allow saliva to go in and come out of such collection chamber. An optical sensor 19 assesses the degree of filling of such collection chamber 15; the valve 16 is opened by a difference of pressure generated by the micropump 18 connected to the collection chamber 15 in an inherently known manner whenever the accessory 3 is located in its respective seat 9A.

The collection chamber 15 is vacuum sealed (or set to a pressure to some extent lower than the atmospheric one) by the base unit 21 whenever the electric toothbrush 1 is placed within a seat 22 of such unit. The seat 22 comprises at least one board 23 on which the electric toothbrush 1 rests and comprising a usual induction recharging member 24 for the rechargeable battery 10 (if any) of the electric toothbrush.

Channels or ducts 25 and 26 (shown in dashed lines in FIG. 3) also lead to the seat 22, suitable for taking saliva from the collection chamber 15 of the accessory 3 of the electric toothbrush 1 and for introducing a washing liquid (subsequently removed through the duct 25) and for creating a vacuum (or a depression) therein, through a number of subsequent steps, respectively. Advantageously, such ducts 25 and 26 include members (not shown in the figures, but inherently known), suitable for opening the valves 16 and 17 placed in such collection chamber 15 whenever the electric toothbrush is placed in the seat 22.

The base unit 21, besides recharging the batteries of the electric toothbrush 1, also includes internally thereto a device 30 suitable for allowing to test a user's saliva. The component parts of such device 30 are schematically shown in FIG. 4.

More specifically, the device 30 (also referred to as “cartridge” in that it is either a disposable element or capable of performing a number of tests before being replaced) comprises a test chamber 31 accommodating a test element 32 complete with one or several inherently known electrodes and receiving the saliva picked up from the collection chamber 15 via a channel or duct 25A; an electric pump 33 is connected to a distribution valve 36 (which the ducts 25 and 26 lead to and the duct 25A departs from), via a duct 35, and said valve picks up one or several reagents from corresponding tanks or containers. In FIG. 4, there are three tanks (37, 38, and 39), and an equal number of ducts 37A, 38A and 39A, connected to such valve 36, depart therefrom.

A duct 40 connected to a tank 41 containing a reference solution also leads to the electrodes 32 (of an inherently known type for performing a saliva test), and a duct 42 departing from such board terminates in a tank 44 where whatever had been tested by the electrodes of the element 32 is discharged.

The cartridge 30, besides making it possible to test saliva, can also be taken advantage of to wash the taking channel or duct 25 and the collection chamber 15 of the toothbrush 1, also via the valve 36 and the channel or duct 25.

The base unit 21 also comprises usual means (a vacuum pump or the like, not shown) for allowing to create vacuum (or a depression) in the collection chamber 15 of the electric toothbrush 1 after washing it.

The device 30 is connected to a control unit 50, which is secured in the unit 21 and whose features can be updated, suitable for monitoring and controlling the operation of all members of the device itself and also suitable for detecting whether any tank (37, 38 and 39 and 41) gets empty or whether the tank 44 is full. This takes place via usual sensor means (not shown) connected to such control unit 50.

The base unit 21 performs a saliva test (by way of a usual test instrument 51 which the chamber 31 belongs to, connected to the unit 50 and schematically shown in FIG. 4) and, when such test ends, can inform a user (via a display 53) or a server or a remote data collection device wirelessly (preferably in encrypted mode) accordingly.

Preferably, other data coming from other (medical or non-medical) devices related to one and the same user might also be sent to said server or remote data collection device. Such data is processed via a Machine Learning Analysis. The unit 21 (which the receiver 20 receiving the fingerprint data from the sensor 11 of the electric toothbrush is connected to) also provides general information to such server, including: the number of remaining test that can be made (as a function of the status of the cartridge 30 or the filling degree of the tanks 37, 38, 39, 41, and 44), any system malfunctions, and other information, such as for instance the status of the battery of the toothbrush.

The saliva test data assessed by the base unit 21 might also be accessed by a doctor in charge of evaluating such data in order to diagnose the patient's health status.

Note that the tests possibly made by using this combined (taking/testing) system are preferably those suitable for identifying endocrine, immunological, inflammatory, infective, etc. condition markers. In particular, a measurement of cortisol (Cushing Syndrome), a hormone produced by adrenal glands, glucose (diabetes mellitus), of a toxicological type, α-amylase (pancreatic or renal diseases) C-reactive protein (CRP, myocardial infarction), total proteins α-amylase), immunoglobulins, Lysozymes, Cystatins, Albumins, Histatins, Lipases, Mucins), pH, in addition to the already known tests (HIV, Fertility test, Pregnancy Test). The movement sensor 13 (also connected to the base unit 21), can also be used to determine whether diseases like the Parkinson's disease occur or get worse, by measuring acceleration.

The use of a system according to the invention will be now described with reference to FIGS. 3 thru 5.

Let's assume to pick-up an electric toothbrush 2 from the base unit 21. With reference to FIG. 5, the procedure of use of the invention includes an initial step 60 followed by a recognition of a user's fingerprint 61, which recognition is made through the use of the sensor 11. If such recognition does not take place or is not confirmed (block 62), then it's back to block 60; otherwise, it goes to block 63 and the valve 16 is opened (block 64), which allows to collect a user's saliva (block 65). The valve 16 opens upon recognizing a user and a predetermined portion of the electric toothbrush in the space (horizontality and insertion of the accessory 3 of the toothbrush in the oral cavity) via the data detected by the sensor 13. If the valve 16 does not open for a reason whatsoever, then an audible alarm signal is generated (block 66).

Note that the introduction of saliva in the collection chamber 15 takes place automatically thanks to the latter being at a pressure lower than the atmospheric one.

Then, an alarm signal is generated (block 68) if the optical sensor 19 does not detect a minimum filling level of the collection chamber 15 (block 67) and in the case the user disconnects the rotary body 4 from his/her own mouth or activates such body 4 before the collection chamber 15 is full.

If the saliva sample has been collected correctly, then the valve 16 is closed (block 69) and the user receives a message (audible indicator or displays 53 in the base unit 21), as indicated by block 70.

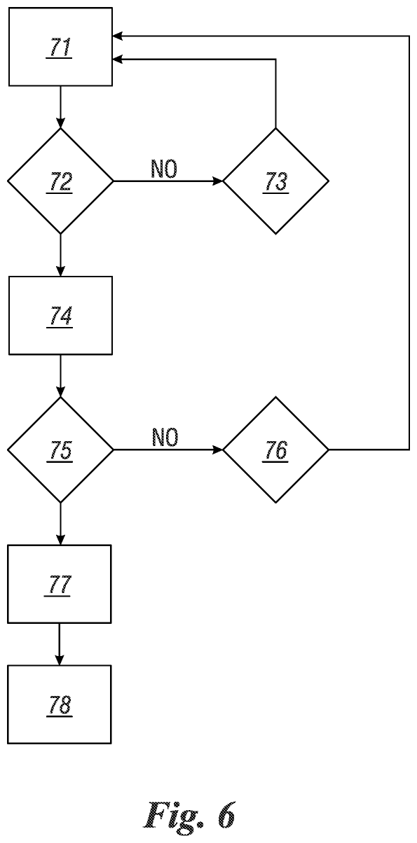

Once the user ends using the toothbrush for cleaning his/her teeth, then this is put again in its seat 22 and a sample test starts according to the procedure illustrated in FIG. 6. After an initialization step (block 71), a sensor located in the seat 22 (not shown) detects the correct positioning of the toothbrush (block 72) in order to be able to allow a connection of the collection chamber to the duct or channel 25. If such positioning is wrong, then an audible alarm signal is generated (block 73).

If the positioning is correct, then the valve 17 is opened (block 74) and saliva is sucked via the pump 33 (block 75). If this does not take place, then an audible alarm signal is generated (block 76).

The sucked saliva is then transferred from the collection chamber 15 to the test element 32 (block 77) and a subsequent substantial sample test can start, whereas the saliva taking procedure ends (block 78).

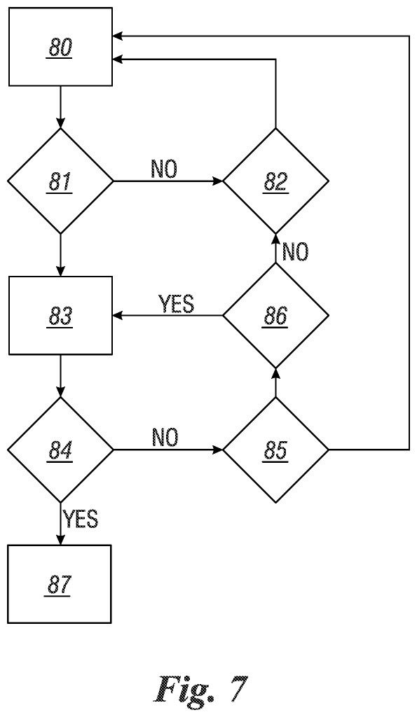

The substantial saliva test procedure is illustrated in FIG. 7: after an initialization step (block 80), a check is made to see whether the saliva sample has been correctly imported into the test chamber 31 or in the test element 32 (block 81). If this is not the case, then an audible alarm signal is generated (block 82).

Then a saliva test is performed (block 83) and the possibility of providing the results of such test is evaluated (block 84). If such possibility does not exist (for instance, because of the test element 32 not being calibrated), then an audible alarm signal is generated (block 85) and the procedure starts again from block 80 (if the quantity of saliva to be tested is not sufficient any longer) or alternatively a check is made to see whether the quantity of the saliva sample is sufficient (block 86) and a test is repeated.

If there is no possibility of getting results, then the substantial sample test ends (block 87).

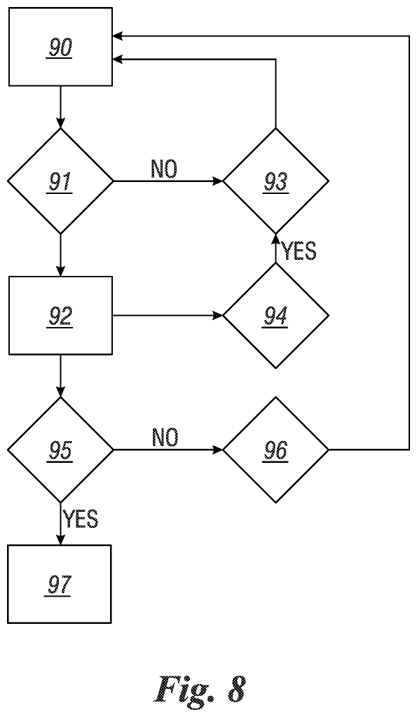

When the test is over, the test chamber, i.e. the electrodes of the test element 32 and of all channels which saliva passed through, is rinsed (FIG. 8). The procedure starts with an initialization step (block 90) and goes on with rinsing (block 91). If the latter is performed correctly, then whatever was removed from the test chamber 31 is discharged into the tank 44 (block 92). If rinsing was not performed correctly, then an alarm signal is generated (block 93) and the procedure is repeated. Note that a check is also made during the discharge procedure to see whether the tank 44 is full or not (block 94) and it's back to block if such filling condition is found 93.

After discharge, a check is made to see whether the test chamber 31 is effective through a calibration of usual sensors present therein, not shown in the figures (block 96) and, if any problems are found, an audible alarm is activated (block 95) and the procedure is repeated from the setting step. If the calibration check is successful, then the procedure ends (block 97).

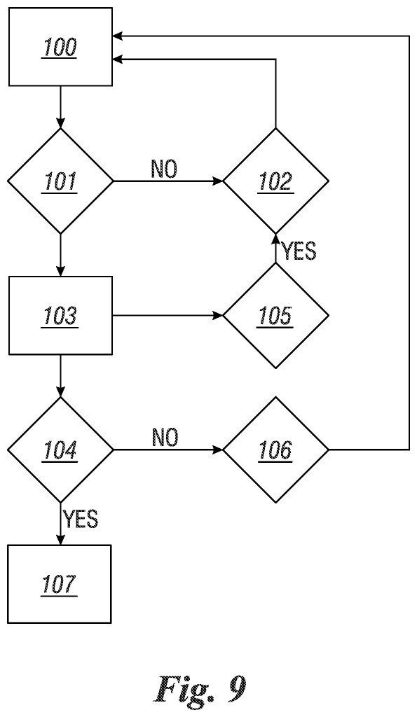

The use of the system according the invention also includes a step of rinsing the collection chamber 15 of the electric toothbrush 1 and the creation of a vacuum therein. This is illustrated in FIG. 9 where, after an initialization (block 100), the actual cleaning of the collection chamber 15 is made and checked (block 101). Should it be found that such cleaning was not made correctly (block 102), then it's back to block 100; otherwise, it goes to the step of discharging the collection chamber 15 (block 103) and creating a vacuum therein (block 104). In this case too, a check is made to see whether the tank 44, which receives the liquid used to rinse the collection chamber 15, is full (block 105). If affirmative, it goes to block 102.

If vacuum generation is not performed correctly in the collection chamber 15, then an alarm signal is generated (block 106) and it's back to the start of the procedure. If everything has been performed correctly, then the procedure ends and the toothbrush 1 can be re-used.

Note that all alarm signals are generated by the base unit 21 via a display 53.

According to the invention, the system comprises saliva collection means (electric toothbrush) and test means (base unit 21) that are physically separate from each other, thus introducing a system scalability advantage, being it possible to add and/or vary the number and type of tests one wishes to perform. Also, the recognition, being made on the toothbrush via a sensor 11, introduces a necessary verification of the patient's ID for a unique association of the test results with a patient. Advantageously, the same sensor also detects the heartbeat and the blood flow while the saliva sample is being collected. Saliva sample tests are performed at a time subsequent to the collection one, thus preventing patients from being obliged to wait for tests to be completed.

In one variant, the testing system is connected to a server in a protected (or encrypted) wireless mode, wherein the server collects the test results sent by various data collection devices. The purpose of the system is also to collect and aggregate all pieces of information uniquely bound to one and the same patient ID, besides the data coming from other sources (clinics, other users, universities, research centers) and to create specific guidelines for each individual user via Machine Learning algorithms which exploit artificial intelligence.

A user that uses this system is either:
A. A healthy person who, via this system, receives information on which lifestyle he/she should adopt to continue to remain “healthy”, or
B. A patient who already suffers from a disease, but shall not get worse or has even the possibility of recovering, or
C. A chronic patient who wishes to extend his/her life expectance as much as possible.

Machine Learning is a data analysis method that builds up analytical models automatically. Machine Learning uses known algorithms that learn from data iteratively to enable systems to identify unknown information, without being it explicitly programmed where to seek it.

More characteristics are collected (preferably from a day-by-day analysis), more quickly the system learns from the collected data via Machine Learning.

In one further and simplified embodiment of the invention, the Machine Learning technique can obviously be used in the base unit 21 by the control unit 50, which can store the data received from the tests performed in a predetermined period of time and, if necessary, transfer it to a remote device, such as a smartphone or an encrypted access web site or others, upon a user's request.

Also, the control unit 50 can be remotely updated, for example via web or via a smartphone, in order to evaluate new test parameters.

The system according to the invention is easy to use and is based on usual user's behaviors; therefore, the system operates without any need for such user or patient to strive to modify his/her habits, and consequently its use is easy and effective.

1. A saliva collection and testing system comprising an electric toothbrush (1) suitable for getting in contact with a user's saliva, said toothbrush comprising a base portion (2) which a preferably separatable accessory (3) is associated with including a usual rotary body (4) with bristle (5), the base portion (2) containing a motor (7) for driving said rotary body (4) into rotation, a test unit (21) being provided for testing such saliva, separate from the toothbrush and suitable for co-operating with such electric toothbrush (1), whenever the latter is coupled with such test unit, the electric toothbrush (1) comprising means (15) for taking a user's saliva, said test unit (21) including a seat (22) for containing the electric toothbrush (1) and comprising means (25, 33) for removing the saliva collected by the electric toothbrush (1) and test means (31, 32, 51) for testing the taken saliva, said test unit (21) cleaning-up the saliva taking means (15) of the electric toothbrush (1) after saliva is taken so as to prepare said electric toothbrush (1) to a subsequent utilization, the toothbrush comprising recognition means (11) for recognizing a user defined by a digital fingerprint sensor (11), said sensor being remotely connected to the test unit and enabling said test unit (21) to couple the reading of a digital fingerprint with a specific user previously stored in such base unit (21) and to have a unique and automatic association of the taken saliva with the user characterized in that said test means of the test unit (21) comprise a test chamber (31) including at least one electrode suitable for receiving the saliva taken by the electric toothbrush (1), a duct (25A) being provided within said test unit (21) functionally connected to a pump (33) suitable for taking such saliva from the electric toothbrush (1) and transferring it into the test chamber (31), a saliva test device (51) being connected to the latter, said test chamber (31), said pump, and said test device being part of an element (30) separatable from the test unit (21) and replaceable.

2. The system according to claim 1, characterized in that said recognition means (11) also allow to measure a user's heartbeat and blood flow, hence his/her blood pressure.

3. The system according to claim 1, characterized in that the toothbrush comprises a movement sensor (13) suitable for assessing the movement, i.e. the acceleration and the position in the space of the electric toothbrush (1) with respect to the test unit, said movement sensor being remotely connected so said test unit (21) and being provided in the base portion (2) of the toothbrush.

4. The system according to claim 1, characterized in that the digital fingerprint sensor (11) is placed in said base portion (2).

5. The system according to claim 1, characterized in that the means used for taking a user's saliva are a collection chamber (15) for collecting said saliva implemented close to the rotary portion (4) of such electric toothbrush, closing valves (16, 17) being associated with such collection chamber (15), said collection chamber being held to an internal pressure at least lower than the atmospheric one until the opening of one (16) of such valves (16, 17), such opening sucking the user's saliva within said collection chamber (15).

6. (canceled)

7. The system according to claim 5, characterized in that a first duct (25) connected to the pump (33) opens in the seat (22) for the electric toothbrush at a first (17) of the closing valves (16, 17) associated with the collection chamber (15) of the electric toothbrush (1), a second duct (26) of said test unit (21) opens in said seat at a second (16) of said closing valves (16, 17), said ducts (25, 26) of said test unit (21) being provided with members for the automatic opening of such closing valves (16, 17) whenever the electric toothbrush is in said seat (22).

8. The system according to claim 1, characterized in that the test unit (21) comprises at least one tank (37, 38, 39) containing a corresponding reagent for performing a saliva test and a tank (44) for receiving the tested saliva, said tanks (37, 38, 39, 41, 44) being part of the separatable element (30).

9. The system according to claim 5, characterized in that it comprises means for rinsing the saliva collection chamber (15) of the electric toothbrush.

10. The system according to claim 5, characterized in that it comprises means for generating at least a depression within the saliva collection chamber (15) of the electric toothbrush.

11. The system according to claim 1, characterized in that the saliva test unit (21) is connected to an Internet server suitable for collecting data obtained from the test of the saliva taken by a user from an electric toothbrush, said Internet server operating according to the Machine Learning procedure.

12. The system according to claim 1, characterized in that the saliva test unit (21) includes a display member for displaying to the user the data obtained from the analysis of the taken saliva.

13. The system according to claim 1, characterized in that the saliva test unit (21) comprises a control unit (50) for controlling the assessed data.

14. A saliva collection and testing system comprising an electric toothbrush (1) suitable for getting in contact with a user's saliva, said toothbrush comprising a base portion (2) which a preferably separatable accessory (3) is associated with including a usual rotary body (4) with bristle (5), the base portion (2) containing a motor (7) for driving said rotary body (4) into rotation, a test unit (21) being provided for testing such saliva, separate from the toothbrush and suitable for co-operating with such electric toothbrush (1), whenever the latter is coupled with such test unit, the electric toothbrush (1) adapted to take a user's saliva, said test unit (21) including a seat (22) for containing the electric toothbrush (1), said test unit (21) cleaning-up the saliva taking means (15) of the electric toothbrush (1) after saliva is taken so as to prepare said electric toothbrush (1) to a subsequent utilization, the toothbrush comprising a digital fingerprint sensor (11), said sensor being remotely connected to the test unit and enabling said test unit (21) to couple the reading of a digital fingerprint with a specific user previously stored in such base unit (21) and to have a unique and automatic association of the taken saliva with the user characterized in that the test unit (21) comprises a test chamber (31) including at least one electrode suitable for receiving the saliva taken by the electric toothbrush (1), a duct (25A) being provided within said test unit (21) functionally connected to a pump (33) suitable for taking such saliva from the electric toothbrush (1) and transferring it into the test chamber (31) for testing, said test chamber (31) and said pump, and said test device being part of an element (30) separatable from the test unit (21) and replaceable.
